(19) United States
(12) Patent Application Publication
(10) Pub. No.: US 2003/0176797 A1
(43) Pub. Date: Sep. 18, 2003

(54) Thrombust; implantable delivery system sensible to self diagnosis of acute myocardial infarction for thrombolysis in the first minutes of chest pain

(76) Inventor: Fernando Anzellini, Bogota (CO)

Correspondence Address: Fernando Anzellini, Calle 83 No 19-36 Office 704, Bogota, CO

(21) Appl. No.: 10/094,981

(22) Filed: Mar. 12, 2002

(51) Int. Cl. A61B 5/0402

(52) U.S. Cl. 600/509

(57) ABSTRACT

A system for recognizing Acute Myocardial Infarction through a portable device (8) and an implantable pump (13) by the patient himself in order to be able to trigger a signal for delivery of drug after symptoms arise, with the advantages of an early thrombolysis without the help of medical doctors or technicians is described in order to install treatment as soon as possible.

The device (8) is capable of warning the user that he/she may be suffering a heart attack so that a signal can be triggered to activate a previously subcutaneous implanted pump (13) filled with the necessary drug for immediate thrombolysis, or any other treatment currently available, and influenced by early diagnosis of chest pain symptoms, this being a worldwide medical problem since Acute Myocardial Infarction is the leading cause of mortality in the world.

### CROSS REFERENCE TO RELATED APPLICATIONS

Related to the present invention is the existence of prior art as illustrated in U.S. Pat. No. 6,339,720 by Anzellini et al. called the CARDIOST, in the form of a unit capable of informing a user with no medical background when he/she is suffering an Acute Myocardial Infarction within minutes of chest pain.

### BACKGROUND FIELD OF INVENTION

The THROMBUST relates to an implantable system that after having meet the criteria for diagnosis of Acute Myocardial Infarction triggers a signal through the skin to a previously implanted subcutaneous pump in order to deliver thrombolysis or any other available treatment, to the body within minutes through a catheter

### BACKGROUND DESCRIPTION OF PRIOR ART

There are many implantable drug delivery systems that delivers drugs from a reservoir at a controllable rate using a pump to impel a drug through a catheter. Localized drug delivery has become increasingly important in applications such as tissue engineering, growth regulation, pain control and therapeutic approaches to localized disease conditions including tumors, local nervous system conditions and local vascular conditions. The action of many drugs is greatly enhanced by, or in some cases requires long term local delivery of those drugs into the Patient's body. Implantable drug delivery devices, implantable pumps, for example have been developed to address the disadvantage of techniques that use external pump and catheter systems. Implantable drug delivery pumps often include a reservoir for storing the drug, an injection port to enable injection of fresh drug preparations at regular intervals into the reservoir, and optionally a catheter for delivering the drug to the desired site. There is the need to inject substances within the body in the exact moment the event is taking place as in Acute Myocardial Infarction when pain arises and the S-T segment of the electrocardiograph shifts either positive or negative, the sooner this is accomplished the better the prognosis. The signal received and analyzed by the CARDIOST U.S. Pat. No. 6,339,720, without the help of qualified medical personal and done by the Patient himself in the moment of chest pain in virtually any environment is then used to trigger a signal to an implantable drug delivery pump in order to provide the necessary amount of available drug in the exact moment it is needed, provided that the prognosis of Acute Myocardial Infarction, that is the leading cause of death in the world, is greatly influenced by early treatment.

U.S. Pat. No. 4,013,074 issued to Inventors: Siposs; George G. (2855 Velasco Lane, Costa Mesa, Calif. 92626) on Mar. 22, 1977

This is an implantable medication-dispensing device which is arranged for subcutaneous implantation. The device has hard body in which is located a liquid reservoir, an adjacent gas chamber, and a pump that which is manually operable through the cutaneous layer for drawing liquid medication from the reservoir and dispensing it and directing it into the body tissue below the hypoderm. A refill membrane permits injection of medication into the reservoir so that the implanted device can be periodically refilled. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 4,692,147 issued to Inventors: Duggan; Stephen R. (Rosemount, Minn.) Assignee: Medtronic, Inc. (Minneapolis, Minn.) on Sep. 8, 1987

In accordance with one aspect of this invention, there is provided a drug administration device for providing a measured dosage of a medical liquid to a location within the body comprising a pump for metering a measured amount of drug from said device in response to a pulse, a triggerable circuit for generating a pulse and a digital computer operable at predetermined intervals to trigger said circuit to generate a plurality of pulses to deliver measured drug dosages at selected intervals.

The device is programmed as to dosage and interval by an external programmer. A “especial” external programmer can reprogram an implanted device to alter its “personality” ie. the duration of the stepper motor drive pulses, the delay time between pulses and the number of pulses to be dispensed for a dosage command can all be altered. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 5,207,666 issued Inventors: Idriss; Samir F. (Hyde Park, Mass.); Makower; Joshua (Nanuet, N.Y.) Assignee: Infusaid, Inc. (Norwood, Mass.) on May 4, 1993

A fluid metering device useful in implantable drug delivery systems is coupled between a pressurized fluid source and an outlet conduit to provide discrete flow pulses at a predetermined rate. The metering device consists of a first movable member constrained within a housing and a second movable member retained in a cavity by the first. Alternate positioning of the first member simultaneously fills and empties the cavity in which the second resides, discharging fluid through the conduit. The first movable member can be a planar slider, an axially translatable cylinder or a rotatable cylinder. The second movable member may be a flat shuttle, a sliding bar or a diaphragm. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 5,443,459 issued to Inventors: Wong; Patrick S. L. (Palo Alto, Calif.); Theeuwes; Felix (Los Altos Hills, Calif.); Larsen; Steven D. (Dublin, Calif.); Dong; Liang C. (Mountain View, Calif.) Assignee: Alza Corporation (Palo Alto, Calif.) on Aug. 22, 1995

The present invention is directed to a fluid-imbibing dispensing device for the initially delayed delivery of an active agent to a fluid environment of use and to a method of using the dispensing device. The dispenser comprises a housing having a first wall section and a second wall section in reversibly sliding telescopic arrangement with each other, which housing maintains its integrity in the environment of use; an internal compartment surrounded and defined by the housing; at least one active agent formulation in the compartment; and expansion means and a push plate in the compartment for separating apart the first and second wall sections of the housing after exposure to the environment of use to expose the active agent formulation to the environment of use. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 5,498,255 issued to Inventors: Wong; Patrick S.-L. (Palo Alto, Calif.) Assignee: Alza Corporation (Palo Alto, Calif.) on Mar. 12, 1996

The present invention is directed to a fluid-imbibing dispensing device for the extended or protracted delivery of an active agent following an initially delayed startup of the delivery to a fluid environment of use. The dispenser comprises a housing having a first wall section and a second wall section in reversibly sliding telescopic arrangement with each other, which housing maintains its integrity in the environment of use; an internal compartment surrounded and defined by the housing; at least one active agent formulation in a portion of the compartment defined by the first wall section; at least one opening in the side wall of the first wall section, each opening extending longitudinally along a portion of the side wall of the first wall section for providing communication between the active agent formulation and the environment; expansion means within a portion of the compartment defined by the second wall section, for separating apart the first and second wall sections of the housing after exposure to the environment of use; and, optionally, a partition layer or push plate between the active agent formulation and the expansion means. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 5,531,736 issued to Wong; Patrick S.-L. (Palo Alto, Calif.); Theeuwes; Felix (Los Altos Hills, Calif.); Larsen; Steven D. (Dublin, Calif.); Dong; Liang C. (Mountain View, Calif.) Assignee: Alza Corporation (Palo Alto, Calif.) on Jul. 2, 1996

The present invention is directed to a fluid-imbibing dispensing device for the initially delayed delivery of an active agent to a fluid environment of use and to a method of using the dispensing device. The dispenser comprises a housing having a first wall section and a second wall section in reversibly sliding telescopic arrangement with each other, which housing maintains its integrity in the environment of use; an internal compartment surrounded and defined by the housing, at least one active agent formulation in the compartment; and expansion means and a push plate in the compartment for separating apart the first and second wall sections of the housing after exposure to the environment of use to expose the active agent formulation to the environment of use. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 5,752,930 issued to Inventors: Rise; Mark T. (Monticello, Minn.); Baudino; Michael D. (Coon Rapids, Minn.) Assignee: Medtronic, Inc. (Minneapolis, Minn.) on May 19, 1998

An implantable catheter defining a plurality of fluid exits responsive to a first range of pressure on a fluid agent for delivering substantially the same flow rate of the agent through each of the exits into spaced infusion sites and responsive to a second range of pressure less than a threshold pressure on the agent for inhibiting the flow of the agent from any of the exits into any of the sites. A first pressure in the first range is applied to the agent in the catheter for a first time period and a second pressure in the second range is applied for a second time period to the agent in the catheter so that substantially the same dosage of agent can be applied to each of the sites. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 5,820,589 issued to Inventors: Torgerson; Nathan A. (White Bear Lake, Minn.); McMullen; Raymond F. (Shorewood, Minn.) Assignee: Medtronic, Inc. (Minneapolis, Minn.) on Oct. 13, 1998

In accordance with the present invention, an implantable pump is programmed non-invasively by means of a programmer that communicates flow rate information by means of radio frequency telemetry or other methods of non-invasive telemetry. The programmer also supplies power to the implantable pump during programming. The implanted rate-adjustable pump that receives rate information and power by telemetry preferably does not include a battery or any other type of internal power supply, relying only on the energy obtained from the programmer through telemetry. Thus, a non-invasively rate-adjustable pump in accordance with the present invention has an advantage over prior single rate restrictor pumps in that a new target rate can be selected non-invasively after the pump has been implanted by changing the state of a valve or valves. In our invention there is no need to supply power as we assume that the need for the latter is scarce because the drug will be injected sporadically. The device is not sensible to telemetry commando orders of a transmitted signal triggered by ST segment shifts either positive or negative measured in real time, that are present in the presence of Acute Myocardial Infarction, but is sensible to telemetry orders in order to change the infusate flow rate to deliver the necessary drug to the blood within minutes for thrombolytic action or any other currently available therapeutic system needed for rapid deliver of drugs

U.S. Pat. No. 5,836,935 issued to Inventors: Ashton; Paul (75 Chestnut St., #13, Boston, Mass. 02108); Patchell; Roy A. (442 Fayette Park, Lexington, Ky. 40508); Cooper; Jon (P.O. Box 55552, Lexington, Ky. 40555); Young; Byron A. (2040 Von List Way, Lexington, Ky. 40502) on Nov. 17, 1998

An implantable, refillable, rate-controlled drug delivery device, with a hollow reservoir, and a drug delivery tube communicating with the hollow reservoir. The drug delivery tube includes at least one rate-limiting permeable membrane which regulates drug delivery. A method of controlling the delivery of a drug to an internal portion of a body is disclosed. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 5,843,069 issued to Inventors: Butler; Mark D. (Flagstaff, Ariz.); Davidson; Daniel F. (Flagstaff, Ariz.); Mish; Stanley L. (Flagstaff, Ariz.); Moore, III; James W. (Flagstaff, Ariz.) Assignee: Gore Hybrid Technologies, Inc. (Flagstaff, Ariz.) on Dec. 1, 1998

This invention relates generally to an implantable containment apparatus made of selectively permeable material. In particular, the implantable containment apparatus is used to contain a therapeutical device, such as a drug delivery device, a cell encapsulation device, or a gene therapy device. A therapeutical device can be easily placed and replaced in an apparatus of the present invention without damaging tissues associated with the selectively permeable material of the apparatus. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 5,976,109 issued to Inventors: Heruth; Kenneth T. (Maple Grove, Minn.) Assignee: Medtronic, Inc. (Minneapolis, Minn.) on Nov. 2, 1999

The present disclosure describes a system wherein a drug or other fluid to be delivered to a specific desired location within the body is stored in a reservoir that is directly displaced by a force to infuse the drug from the device into the patient. Several specific methods are used to displace the reservoir, including, generally, hydraulic displacement, mechanical screw-type displacement, and spring force displacement of the fluid reservoir. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 5,061,242 issued to Inventors: Sampson; Edward J. (Carlisle, Mass.) Assignee: Infusaid, Inc. (Norwood, Mass.) on Oct. 29, 1991

A drug delivery system for implantation into a living body having a pressure actuated infusion pump and an adjustable flow regulator, The regulator comprises a body having a regulator cavity divided by a diaphragm into two chambers. One chamber serves as a pressure sensor and the other as a conduit through which fluid flows. The outlet from the conduit chamber can be sealed by diaphragm movement as a function of pressure increase or the flow restricted. The outlet itself is mounted on an adjustable fitting that is movable relative to the diaphragm. The distance between the outlet and the diaphragm may be set as a calibration of the regulator. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 4,714,462 issued to Inventors: DiDomenico; Robert A. (Norfolk, Mass.) Assignee: Intermedics Infusaid, Inc. (Norwood, Mass.) on Dec. 22, 1987

A positive pressure positive displacement implantable infusion pump. A positive pressure fluid reservoir maintains infusate for delivery by a positive pressure displacement pump. Infusate is drawn by the pump into a drug pressurant chamber. A check valve prevents backflow into the reservoir. The check valve may independently establish a basal flow rate by not completely sealing in the forward flow direction. The pump pulses to expel and fill the chamber thereby establishing the flow rate, basal or bolus through a restrictor interposed between the chamber and the infusion site. The pump may run in open loop, pre-programmed or externally programmed modes of operation. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 4,931,050 issued to Inventors: Idriss; Samir F. (Mattapan, Mass.) Assignee: Shiley Infusaid Inc. (Norwood, Mass.) on Jun. 5, 1990

A variable flow constant pressure implantable pump employs an in-line needle restrictor having a variable insertion distance in a capillary. The degree of insertion determines the effective restriction within the capillary. The restrictor may be repositioned following implantation to vary the drug delivery rate by changing the effective restriction in the capillary. Various couplings may be used such that an auxiliary or bolus port delivers infusate directly into the outlet catheter so that medication from the main chamber does not pass through the auxiliary chamber. A combination of double lumen catheters and T-connectors are employed to provide parallel infusion paths. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 4,077,405 issued to Inventors: Haerten; Rainer (Erlangen, DT); Kresse; Heinz (Erlangen, DT) Assignee: Siemens Aktiengesellschaft (Berlin & Munich, DT) on Mar. 7, 1978

Apparatus for feeding liquids, particularly medications such as insulin, or the like, to humans or animals comprising a supply reservoir for the liquid, a conveying arrangement connected to the supply reservoir and including a pressure generating device for feeding the liquid by pressure application from the supply reservoir to the body, and a controllable dosing arrangement for producing a desired rate of infusion. The pressure generating device includes an overpressure generator which constantly maintains the liquid present in the supply reservoir under overpressure as distinct from the pressure prevailing at the point of liquid discharge. The dosing arrangement comprises a pilot or switching valve for periodically releasing liquid from the supply reservoir to the body, the switching valve being switchable for the purpose of fine dosing by the discharge of preselectable constant volumes of liquid at periodic intervals predeterminable in accordance with the treatment program. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 4,443,218 issued to Inventors: DeCant, Jr.; Leonard J. (Allston, Mass.); Idriss; Samir F. (Arlington, Mass.) Assignee: Infusaid Corporation (Norwood, Mass.) on Apr. 17, 1984

A programmable implantable infusion pump includes a housing containing a variable volume infusate chamber and variable volume control fluid pressure and displacement reservoirs. A conduit conducts infusate from the chamber exteriorly of the housing. A restricted fluid path exists between the two reservoirs which are filled with an incompressible control fluid. A loaded spring applies a constant force to the pressure reservoir causing fluid flow between the two reservoirs. The differential pressure across the flow restriction is sampled by a microprocessor which computes the fluid flow rate, that rate being proportional to the infusate flow rate from the chamber. When the fluid flow rate deviates from a programmed value, the processor issues an error command to a battery-driven motor which reloads the spring to reestablish the constant force on the pressure reservoir. The infusate flow rate may be changed automatically during the day in accordance with a programmed schedule and that schedule may be changed by reprogramming the processor using a telemetry receiver contained in the housing. The device is not sensible to telemetry commando orders of a transmitted signal triggered by ST segment shifts either positive or negative measured in real time, that are present in the presence of Acute Myocardial Infarction, but is sensible to telemetry orders in order to change the infusate flow rate to deliver the necessary drug to the blood within minutes for thrombolytic action or any other currently available therapeutic system needed for rapid deliver of drugs

U.S. Pat. No. 4,447,224 issued to Inventors: DeCant, Jr.; Leonard J. (Allston, Mass.); Idriss; Samir F. (Arlington, Mass.); Prosl; Frank R. (Duxbury, Mass.) Assignee: Infusaid Corporation (Norwood, Mass.) On May 8, 1984

Implantable variable flow infusion apparatus comprises a collapsible infusate chamber having an outlet conduit leading to the infusion site by way of series-connected fixed and variable fluid flow restrictors. The infusate in the chamber is maintained under substantially constant pressure by a nonelectric energy storage cell to establish flow from the chamber to the site. The variable restrictor is controlled by commands from a preprogrammed microprocessor and programmable memory powered by a small long-lived battery. The processor sets the variable restrictor to the proper setting to establish the correct infusate flow for the particular time of day. A pressure transducer connected across the fixed restrictor senses the pressure differential across the fixed restrictor which differential indicates actual infusate flow from the chamber. The processor samples the output from the transducer at infrequent sampling intervals and issues command signals to control the variable restrictor whenever the actual infusate flow rate differs from the preprogrammed rate so that the apparatus consumes a minimal amount of battery energy and can thus remain implanted in the body for a prolonged period. The processor can also be reprogrammed and monitored by telemetry. The device is not sensible to telemetry commando orders of a transmitted signal triggered by ST segment shifts either positive or negative measured in real time, that are present in the presence of Acute Myocardial Infarction, but is sensible to telemetry orders in order to change the infusate flow rate to deliver the necessary drug to the blood within minutes for thrombolytic action or any other currently available therapeutic system needed for rapid deliver of drugs

U.S. Pat. No. 4,496,343 issued to Inventors: Prosl; Frank R. (Duxbury, Mass.); Sampson; Edward J. (Carlisle, Mass.) Assignee: Infusaid Corporation (Norwood, Mass.) on Jan. 29, 1985

Infusion apparatus includes a housing with an infusate chamber in the housing and means for conducting infusate from the chamber through a restrictive outlet passage to an additional small chamber having an entrance port closed by a self-sealing penetrable septum. An exit tube from the small chamber leads directly to a cannula at the infusion site so that liquid can be injected into the small chamber through its septum and delivered directly to the infusion site without backflowing into the infusate chamber. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No 4,258,711 issued to Inventors: Tucker; Elton M. (Medfield, Mass.); Prosl; Frank R. (Duxbury, Mass.) Assignee: Metal Bellows Corporation (Sharon, Mass.) on Mar. 31, 1981

An infusion apparatus and method employs an infusate reservoir connected by way of a first flow path having a first flow restrictor to a mixing chamber. The reservoir is also connected by a second flow path having a second flow restrictor to the same mixing chamber so that infusate may be conveyed to the chamber at different rates. A normally closed valve is provided in the second flow path so that infusate flows from the two paths supplement one another in the mixing chamber only when the valve is open. The reservoir and chamber volumes, infusate concentration and chamber outlet flow rate are selected to provide an integrated dosage profile suited to the patient.. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

U.S. Pat. No. 4,838,887 issued to Inventors: Idriss; Samir F. (Mattapan, Mass.) Assignee: Shiley Infusaid Inc. (Norwood, Mass.) on Jun. 13, 1989

An implantable valve accumulator pump for the delivery of medication is disclosed. The implantable pump comprises a drug reservoir maintained a constant pressure vapor.

The medication metering assembly comprises a fixed volume accumulator positioned between a pair of valves. The valves alternately open and close to admit medication from the reservoir into the accumulator and to dispense a precise volume spike to an outlet catheter. In order to minimize dead volume and insure complete discharge, the accumulator employs a titanium diaphragm seated in one position by a recessed stop and in the discharge position by a spacer plate having a groove pattern. The unit is externally programmed. The device is not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

Foreigns Patents No GB 2061521A issued to DavisHowell Jenkins May 1981 measures the individual's susceptibility to cardiovascular disorder with a visual indicator displaying one of a series of indications and enabling answers to be entered. Patent SU 1570-709-A issued to Leca June 1990is a human's heart action monitor that measures heart contraction frequency and ST segment shift. Patent 2 315 064 issued to Burchard March 1973 measures ST fall arrhythmia's with continuously selectable prematurity index and intervals, tachy- and bradycardias, in continuously adjustable intervals and/or frequency regions. Patent DD 281 957 A5 issued to Krinke August 1990 determines the time occurrence of R-blips, QRS complexes and P-waves and allows a complex description of irregularity as well as formulation. Patent DE 3633-983 A issued to Wasser October 1986 measures variations in voltage characteristics and provides a high degree of freedom movement; different output signals are provided in dependence on the variation rate. Patent 5-64632 (A) issued to Takashi Suzuki March 1993 enables quick finding of calculating conditions ensuring a better ST deviation trend graph. Patent 405176906 (A) issued to Mutsuo Kaneko July 1993 measures a peak value of ST segment at an arbitrary measuring point displaying successively generated ST trend graphs, so that many derived electrocardiogram waveforms can be recognized easily. This devices are not sensible to telemetry commando orders by means of a radio frequency head disposed proximate to a pump that includes a telemetry antenna or receiver preferably comprising a coil of wire within which a voltage may be induced when the receiver is in the presence of a transmitted signal triggered by ST segment shifts either positive or negative measured in real time, that are present in the presence of Acute Myocardial Infarction, in order to deliver the necessary drug to the blood within minutes for thrombolytic action or any other currently available therapeutic system needed for rapid deliver of drugs

### SUMMARY

The object of the THROMBUST is to provide a complete system capable of instantaneous electrocardiographic diagnosis for Acute Myocardial Infarction and a implantable delivery system for thrombolytic drugs or any other available current treatment completely synchronized by telemetry orders in order to be delivered in the first minutes of chest pain by the patient himself.

Objects and an Advantages

Accordingly, the main object and advantages of the THROMBUST is for a system that allows for a real-time diagnosis, made by the patient himself, or any other person nearby without the necessary medical training, at the time of acute pain, of Acute Myocardial Infarction (AMI) and thus enables him/her to activate an implantable pump with the necessary drug, currently thrombolytics for fibrinolysis, or any other available treatment that is considered necessary for the individual patient by his physician, or previous studies in the medical literature, in which prompt delivery is important. Not to install treatment in the first minutes of acute pain, after diagnosis of Acute Myocardial Infarction (AMI), is one of the major public health problems in the world, as supported by thousands of papers and studies all over the world. Other devices which make automatic diagnosis of Acute Myocardial Infarction need to be read and interpreted by a physician who most of the time is not available when the patient needs the diagnosis and therefore unacceptable delays in installing proper treatment are the rule. Thus the THROMBUST that is capable of early diagnosis of Acute Myocardial Infarction, can be used, in this system, for immediate delivery of the necessary drug, triggering a telemetry signal to a previous implanted subcutaneous pump.

It is yet another object of the THROMBUST to provide a system capable of instantaneous electrocardiographic measurement that can be used easily by any person, preferably the patient himself, even if such person is under the stress of thinking that he/she is having a heart attack, and is designed to help a patient to distinguish between the common symptoms of chest pain and those of Acute Myocardial Infarction, which is usually very confusing and subjective, and then activate in the system the necessary steps to trigger a signal by telemetry through the skin in a previously implanted subcutaneous pump in order to have the drug injected directly into the body within few minutes of symptoms reducing substantially the time to achieve this gold standard for treatment of Acute Myocardial Infarction that helps reduce dramatically the outcome and prognosis of the leading cause of dead.

These and other purposes of the THROMBUST are achieved by means of the embodiment of a portable electrocardiographic measuring and analyzing unit, preferably a small, low-power electronic unit as is described in U.S. Pat. No. 6,339,720 and then with a telemetry command in the electrocardiographic system, that activates the previous implanted pump under the skin of the patient, that responds to this signal, the necessary amount of drug is delivered to the necessary place in the body as soon as the diagnosis of Acute Myocardial Infarction is made, thus avoiding unnecessary delays in this treatment that has been proved completely changes the outcome of heart disease.

There is great public health concern as to an early diagnosis of Acute Myocardial Infarction, given that a large proportion of patients fails to seek medical attention within the first 4 to 6 hours of the onset of chest pain. If proper medical diagnosis and care are provided within this critical period (of the initial 4 to 6 hours), many lives can be saved with the use of thrombolysis drugs or any other treatment that can be furnished upon further investigation. It is for these cases that the THROMBUST system is intended.

Further objects and advantages of our invention will become apparent upon consideration of the drawings and ensuing description.

### DESCRIPTION OF DRAWINGS

In the drawings, closely related figures have the same number but different alphabetic suffixes

FIGS. 1A and 1B show how the THROMBUST portable electrocardiographic device is attached with a negative wire to the right armpit, with a positive/negative wire to the left armpit, and a positive wire to the lower part of the abdomen the hipogastrium. Behind the THROMBUST there is a positive and negative electrode placed in a V4 position at the 6th to 8th intercostal space with mid-clavicular line. These electrodes may be placed against the skin without any gel or conductive substance. It is included a telemetry activation button

FIG. 2A shows a THROMBUST device with the neutral electrode and precordial positive electrode that are intended to be placed in the precordial area V4. It is included a telemetry activation button

FIG. 2B shows the rear of the device and the battery compartment. It is included a telemetry activation button, and the color codes and alarm

FIG. 3A shows an implantable pump with a telemetry receiver incorporated and placed in a subcutaneous pocket that is able to receive a telemetry signal from the THROMBUST.

FIG. 3B shows the implantable schematic representation of an implantable pump with a radiofrequency head, a telemetry antenna a control circuitry and sensors measuring drug volume in the reservoir and a sensor for measuring flow rates.

### REFERENCE NUMBERS IN DRAWINGS

4 common cable

5 positive/negative left armpit cable

6 negative right armpit cable

7 positive abdomen cable

8 thrombust

9 V4 positive electrode

10 neutral electrode

11 telemetry transmitter

12 battery compartment

13 implantable pump

14 telemetry receiver

15 telemetry antenna

16 subcutaneous pocket

17 control circuitry

18 sensors

19 fluid reservoir

20 red alarm light

21 yellow alarm light

22 green alarm light

23 audible tone alarm

24 septum for filling

### DETAILED DESCRIPTION OF THE INVENTION

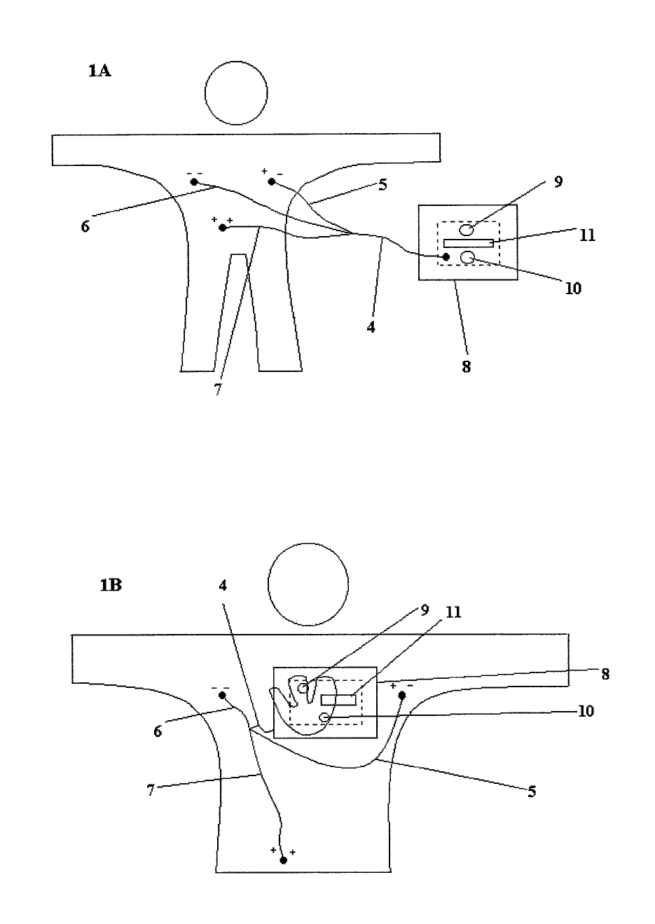

As illustrated in FIG. 1A (top view) and FIG. 1B (end view) a device 8 is comprised of a plastic housing which has a permanently attached electrode cable extending from it, 4, a cable has 3 electrode lead inputs located at the remote end, 5,6,7 and these leads connect to electrodes placed in contact with the patient's body to provide electrical input of his/her electrocardiographic signals. In the right armpit the electrode is negative 6, in the left armpit the electrode is positive/negative 5,and in the lower portion of the abdomen, the hipogastrium, the electrode is positive 7. The telemetry command 11 is included.

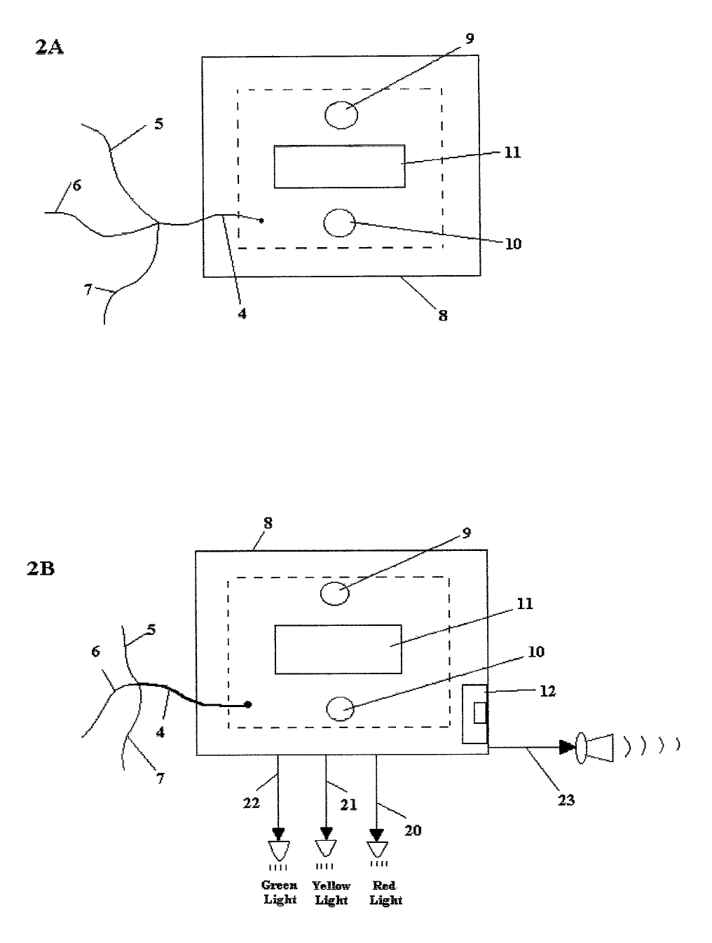

As illustrated in FIG. 2A, at the rear end of a device 8 a positive electrode 9 is used to be placed in the precordial area in V4 to record one electrocardiographic signal that we think is enough for diagnosis of the anterior wall of the heart. A neutral electrode 10 is also shown The telemetry command 11 is included.

As illustrated in FIG. 2B, a battery compartment 12 is located within a device 8, preferably for 1.5 volt alkaline (size AA/AAA) disposable batteries to supply power. A device 8 may be placed in a carrying case, to be used by the patient at any time at night or day, when feeling a chest pain. Since a device may be used immediately at any time the patient will have a high probability of ensuring a timely diagnosis. The telemetry command 11 is included. The electrocardiographic data in real time is analyzed and after processing the information triggers the appropriate alarm green 22 for low risk of Acute Myocardial Infarction (AMI), yellow 21 for medium risk AMI or red 20 and audible tone 23 for high risk of AMI.

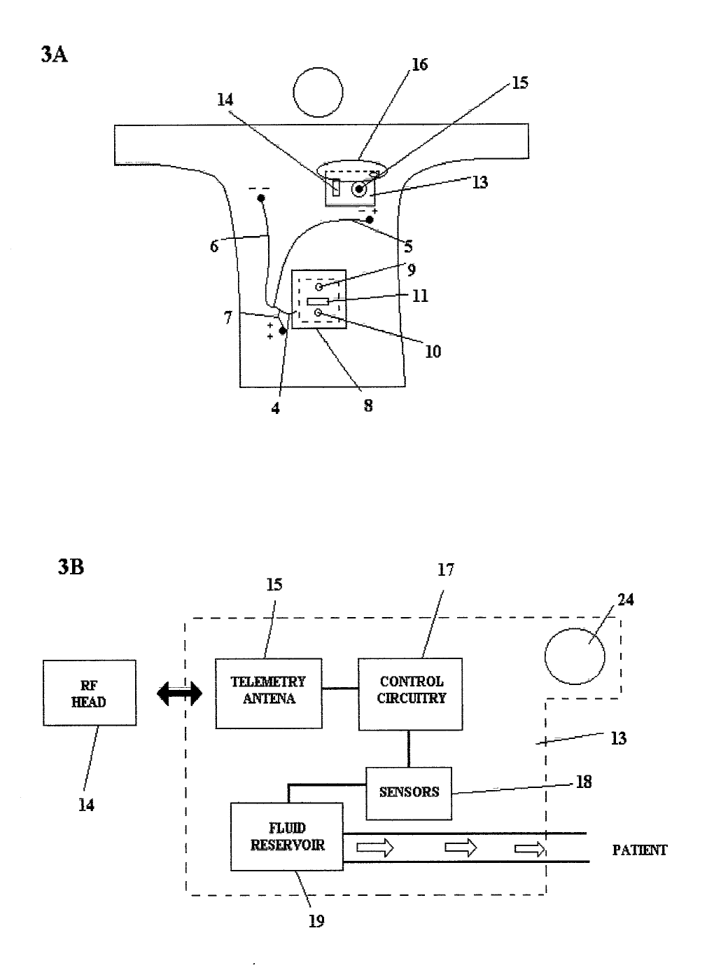

As illustrated in FIG. 3A the device 8 is placed appropriately for diagnosis. Previously in a subcutaneous pocket 16 an implantable pump 13 has been placed that contains a telemetry antenna 15 that responds to a signal from the device 8 when the diagnosis of Acute Myocardial Infarction (AMI) motivates the patient to trigger the signal by means of alarms.

As illustrated in FIG. 3B an implantable pump 13 that contains a telemetry receiver 14 and connects to a telemetry antenna 15 activates a control circuitry 17 and by means of sensors 18 included in the pump 13 a fluid reservoir 19 is opened to deliver the previously stored substance to the patient.

Operation

With the combination of the five electrodes in the device, four active and one neutral, we are able to obtain the standard electrocardiogram derivations for diagnosing Acute Myocardial Infarction in the different areas of the myocardial muscle, i.e.:

(a) Inferior Myocardial Infarction with the electrode combination of D II (right armpit 6 and hipogastrium or positive abdomen cable 7), D III (left armpit 5 and hipogastrium or positive abdomen cable 7) and AVF (left armpit 5 and hipogastrium or positive abdomen cable 7)

(b) Lateral Myocardial Infarction with the electrode combination of AVL (left armpit 5 and hipogastrium or positive abdomen cable 7) and DI (right armpit 6 and left armpit 5)

(c) Anterior Myocardial Infarction with only one electrocardiographic signal obtained from the precordial lead in V4, 9 (intersection of imaginary line of seven to eight intercostal spaces with mid clavicular line).

(d) Posterior myocardial infarction with the mirror image of anterior leads (precordial electrode 9).

An electronic circuit is placed in the housing and is used by the patient by means of acoustic and light signals to assist in the diagnosis of Acute Myocardial Infarction. A green light means low risk, a yellow light means medium risk, and a red light and an audible alarm means high risk of Acute Myocardial Infarction. Thus, when the patient is under medium or high risk, as alerted by the device, he/she can trigger immediately a telemetry signal with a button placed in the THROMBUST in order to activate via a telemetry antenna a fluid reservoir contained in a previously implanted subcutaneous pump so thrombolysis for fibrinolysis or any other medical treatment can be delivered within minutes of chest pain and as fast as possible after the diagnosis. We believe that the treatment of Acute Myocardial Infarction should improve dramatically after our THROMBUST becomes available because is the patient himself (or anyone nearby with or without medical training) that triggers the first steps in achieving thrombolysis, so time, that is a critical factor that influences the outcome of the leading cause of dead, is overcome.

This should be done in the initial minutes of the myocardial infarction onset, when this treatment has an excellent statistically proved outcome in revascularization of the affected infarct zone of the heart. After minutes of chest pain the thrombus starts to organize and as time passes it becomes well established and thrombolysis is used with a lesser probability of success. As said above, with an improved diagnosis of myocardial ischemia, an early diagnosis, the most important factor of ischemia, will be conducive to the development of more specialized treatments so that trombolysis will be merely a part of the initial treatment of ischemia, and soon we will have other therapeutic alternatives, possibly influenced by THROMBUST. It is well known that a very large proportion of patients do not seek medical help in the initial hours of Acute Myocardial Infarction, with a subsequent increase in morbidity and mortality, simply because they are not sure about the nature of their chest pain. We believe that THROMBUST will save many lives in the future, considering that the lack of an early diagnosis of Acute Myocardial Infarction is the leading cause in morbidity and mortality in humans and remains undertreated due to the current lack of procedures for patients to make the right interpretation of their symptoms when pain arises in settings other than a hospital or a doctor's office, which is the case with 99% of the occurrences of Acute Myocardial Infarction.

The device is able to diagnose Acute Myocardial Infarction in any of the different areas of the heart which may be in danger, i.e. inferior, lateral, anterior, and posterior, and any combination thereof.

Conclusions, Ramifications and Scope of the Invention

Thus the reader will see that diagnosis of Acute Myocardial Infarction may be made by the patient himself when chest pain arises, by connecting the device to his body, thus triggering either acoustic or visual alarms This will enable him to trigger a signal to a previous implanted subcutaneous pump for delivery of the drug after symptoms arise, with the advantages of an early thrombolysis or any other treatment available, which will be made possible thanks to an early diagnosis, one of the most difficult problems to be resolved in order to minimize the high morbidity and mortality at present caused by Acute Myocardial Infarction.

Although the above description contains many specificity's, these should not be construed as limiting the scope of the TRHOMBUST but rather as an exemplification of a preferred embodiment thereof.

Many variations are possible. For example, the device may have other types of alarms, other cable connections, may allow for the analysis of different parts of the electrocardiogram with other diagnosis periods, other treatments proposed, analysis of other waves of the electrocardiogram, other signals triggered to activate the previously implanted pump, and many types of pumps that will change and improve and other delivery systems developed to provide drugs within the body.

Thus the scope of the THROMBUST should be determined by the appended claims and their legal equivalents, rather than by the examples given.

1. A method completely managed by the patient himself in the moment of chest pain comprising;
a) a method for self-diagnosis of acute myocardial infarction at the moment of said chest pain indicating ischemic activity for an early diagnosis, in the first minutes of said acute myocardial infarction to determine with a high degree of effectiveness when an artery of the heart is acutely obstructed by thrombus or rupture of atherosclerotic plaque related to said ischemic activity by means of a device capable of sensing a patient's ECG waveform and emitting an audible and visual alarm that triggers a voluntary or automatic response via telemetry, the method comprising;
b) delivering a telemetry signal in the moment of said ischemic activity by the patient himself or other person in the first minutes of said acute myocardial infarction

2. a method for immediate delivery of drugs in acute myocardial infarction by the patient himself or other person in the moment of chest pain comprising;
a) a previously implantable delivery pump that receives the said telemetry signal
b) a said implantable delivery pump providing the necessary amount of drugs after receiving the said telemetry signal in the exact moment of said ischemic activity
